(19) United States
(12) Patent Application Publication
(10) Pub. No.: US 2010/0145421 A1
(43) Pub. Date: Jun. 10, 2010

(54) Therapeutic Cooling and/or Heating System Including A Thermo-Conductive Material

(75) Inventors: C. Mike Tomlinson, Thousand Oaks, CA (US); Robert Croteau, Christiana, PA (US); Mark Lowe, Danville, CA (US); Krister Bowman, Oakland, CA (US); James Orsini, Unionville, PA (US); Andrew W. van Eps, Kennett Square, PA (US)

Correspondence Address: SHAY GLENN LLP, 2755 CAMPUS DRIVE, SUITE 210, SAN MATEO, CA, 94403, US

(73) Assignee: CoolSystems, Inc.

(21) Appl. No.: 12/329,481

(22) Filed: Dec. 5, 2008

(51) Int. Cl. A61F 7/00; F25D 23/00; F28F 7/00; G05D 23/00; F28D 15/00; A61F 7/02

(52) U.S. Cl. 607/104; 62/259.3; 165/46; 165/287; 165/104.19; 607/111

(57) ABSTRACT

In general, the present invention incorporates a cooling or heating wrap having internal channels to circulate a chilled or warmed fluid, with one or more layers of a thermo-conductive material to eliminate air pockets or gaps between the wrap and a human or animal body part. The thermo-conductive material may be applied directly to the human or animal skin and/or body part, may be encapsulated in a separate unit, or may be integrated into the wrap itself. The thermo-conductive material acts like a heat conduction medium between the heat exchanging wrap and the anatomical body part, to increase the therapeutic effect of the wrap.

### BACKGROUND OF THE INVENTION

1. Field of the Invention

The present invention relates generally to therapeutic cooling or heating systems, and more particularly, to a therapeutic wrap of the type having circulating chilled or warm fluid in the wrap to provide cooling, and/or heating, and/or compression to a human or animal body part.

2. Description of the Related Art

Systems are known which provide active cooling and/or compression for humans and other animal bodies. They are used, for example, in physical therapy, pre or post-game conditioning, minor injury care, post-operative care, etc. In general, the body heat exchanging component(s) of such an apparatus has a pair of layers defining a flexible bladder through which a liquid is circulated. This component is often referred to as a “wrap.” The liquid circulated through the wrap is maintained at a desired temperature. Generally, the desired temperature is lower than the temperature expected for the body part, and typically is achieved, at least in part, by passing the liquid through a heat exchanging medium, such as by passing the same through an ice bath, or a refrigeration unit. One such system is disclosed, for example, in U.S. Pat. No. 6,178,562, the disclosure of which is herein incorporated by reference.

The wrap can be constructed, for example, as taught in U.S. Pat. Nos. 6,695,872 and 7,198,093, the disclosures of which is herein incorporated by reference. In addition, a control unit can provide air pressure to the wrap to provide compression, as taught by U.S. Pat. No. 6,695,872.

One issue with these types of therapeutic wraps is that the amount of thermo-conduction is limited and/or inconsistent due to the significant air gaps between the wrap and the body part.

In addition, the use of various “gel packs” are known, which can be frozen and placed on a human or animal body part. The difficulty with these frozen gel packs is that the frozen gel is often difficult to conform to the desired anatomical part, and does not provide for any active cooling—i.e. as the gel melts, the gel pack loses its ability to provide a consistent temperature. One means of improving the gel packs is to develop gels which remain somewhat pliable even when frozen, such as those taught by U.S. Pat. No. 4,925,603. However, such gel packs still do not provide even and consistent cooling for the reasons noted above.

### SUMMARY OF THE INVENTION

In general, the present invention incorporates a cooling or heating wrap having internal channels to circulate a chilled or warmed fluid, with one or more layers of a thermo-conductive material to eliminate air pockets or gaps between the wrap and a human or animal body part. The thermo-conductive material may be applied directly to the human or animal skin and/or body part, may be encapsulated in a separate unit, or may be integrated into the wrap itself.

The thermo-conductive material acts like a heat conduction medium between the heat exchanging wrap and the anatomical body part. The thermo-conductive material can fill in any empty air space between the wrap and provide efficient heat transfer between the two surfaces. Since common body parts have different shapes and dimensions from individual to individual, it is difficult to get a standard wrap to transfer heat effectively. With the improved heat conduction of the thermo-conductive material, the present system provides for better heat transfer, even when used on different sized body parts.

In one embodiment, a therapeutic cooling or heating system for cooling or heating an anatomical body part comprises a wrap configured to conform to an anatomical body part, a cooling or heating unit to cool or heat the wrap, and at least one layer of a thermo-conductive material positioned between the wrap and the anatomical body part.

According to an embodiment of the present invention, a therapeutic cooling or heating system for cooling or heating an anatomical body part, the system comprises a wrap configured to conform to an anatomical body part, the wrap having one or more internal channels for fluid circulation, a cooling or heating unit to provide cooled or warmed fluid to the wrap and to circulate the fluid through the one or more internal channels, and at least one layer of a thermo-conductive material positioned between the wrap and the anatomical body part.

The system may further comprise a temperature sensor located at or near the anatomical body part and a controller connected to receive an output from the temperature sensor, wherein the controller adjusts the temperature of the fluid based on the temperature received from the sensor.

The thermo-conductive material may comprise a gel applied directly to the anatomical body part. The thermo-conductive material can be contained in a protective covering separate from the cooling wrap, or formed as an integral layer of the cooling wrap. The at least one layer of thermo-conductive material may further comprise at least two layers of material, where each layer has a different thermal conductivity and/or abilities to conform (pliability) to the body part.

A method of providing cold or heat therapy to an anatomical body part of the present invention includes placing a first thermo-conductive material on an anatomical body part, placing a wrap around the thermo-conductive material, and cooling or warming the wrap to maintain a constant temperature to the anatomical body part.

According to another method of the present invention, laminitis on a horse may be treated by placing a thermo-conductive material on a horse's foot, placing a cooling wrap around the thermo-conductive material and a lower leg of the horse, and providing a cooling fluid to the cooling wrap, wherein the cooling fluid has a temperature below 5° C.

### BRIEF DESCRIPTION OF THE DRAWINGS

The present invention will be readily understood by the following detailed description in conjunction with the accompanying drawings, wherein like reference numerals designate like structural elements, and in which:

FIG. 1 illustrates an embodiment of a cooling system according to the present invention;

FIGS. 2A, 2B and 2C illustrate embodiments of the present invention in which a thermo-conductive material is formed as a layer integral to a cooling wrap; and

FIG. 3 illustrates an embodiment of the present invention configured to treat laminitis in an equine foot.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention.

In one form, a therapeutic cooling or heating system for cooling or heating an anatomical body part comprises a wrap configured to conform to an anatomical body part, a cooling or heating unit to cool or heat the wrap, and at least one layer of a thermo-conductive material positioned between the wrap and the anatomical body part. For example, the wrap could be a heating or cooling pad fitted around a thermo-conductive material.

In a preferred embodiment, the present invention incorporates a cooling or heating wrap having internal channels to circulate a chilled or warmed fluid, with one or more layers of a thermo-conductive material to eliminate air pockets or gaps between the wrap and a human or animal body part. The thermo-conductive material may be applied directly to the human or animal skin and/or body part, may be encapsulated in a separate unit, or may be integrated into the wrap itself.

The thermo-conductive material acts like a heat conduction medium between the heat exchanging wrap and the anatomical body part. The thermo-conductive material can fill in any empty air space between the wrap and provide efficient heat transfer between the two surfaces. Since common body parts have different shapes and dimensions from individual to individual, it is difficult to get a standard wrap to transfer heat effectively. With the improved heat conduction of the thermo-conductive material, the present system provides for better heat transfer, even when used on different sized or shaped body parts.

While the present invention may be used for both cooling systems, and heating systems, the following descriptions pertain to a cooling system. It is understood that the teachings of the present invention can be applied to therapeutic heating systems as well.

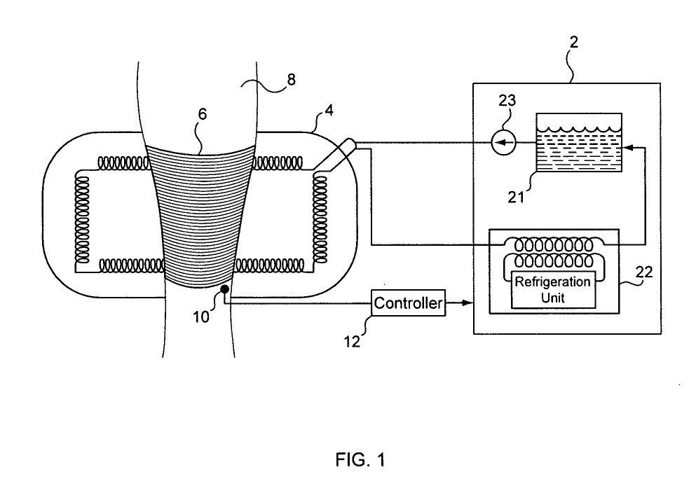

An embodiment of the present invention is illustrated in FIG. 1. A cooling unit 2 contains a fluid reservoir 21, a refrigeration unit 22 to cool the circulation fluid, and a pump 23 to circulate the fluid from the reservoir 21 to a heat exchange cooling wrap 4 (shown open; the wrap in use is wrapped around the body part 8, and is attached together with Velcro, for example).

The cooling wrap 4 can be constructed, for example, as taught in U.S. Pat. Nos. 6,695,872 and 7,198,093, the disclosures of which is herein incorporated by reference. In addition, a control unit can provide air pressure to the wrap to provide compression to the wrap as taught by U.S. Pat. No. 6,695,872.

The cooling unit 2 shown includes a refrigeration unit 22 to provide active chilling of the fluid. For many applications, such as portable human use, the refrigeration unit 23 can be eliminated, and the reservoir simply filled with ice water. For certain animal applications (such as the treatment of equine laminitis, discussed below), the refrigeration unit 23 may chill a heat transfer fluid down to 0° C., or even cooler. Obviously, the heat transfer fluid in this case will have a freezing point below the freezing point of water, as necessary for a particular application.

As noted above, the present invention can also be used to apply heat to anatomical body part, in which case heated fluid is applied to the wrap. The refrigeration unit 23 can simply be exchanged for a heating unit (not shown) in the embodiment of FIG. 1. For certain applications, the system may include both a refrigeration unit and a heating unit, as desired.

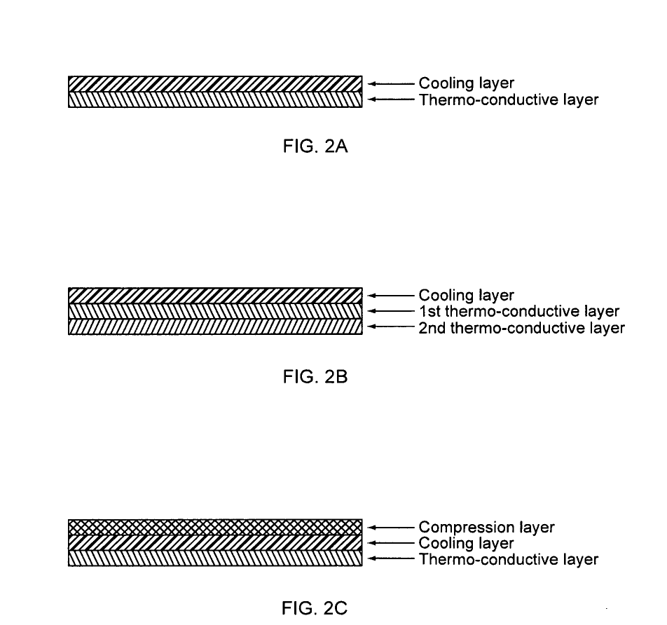

The cooling wrap 4 is placed over a thermo-conductive material 6, which is placed against the anatomical body part 8. The thermo-conductive material 6 effectively provides an improved heat transfer medium to interface the cooling wrap 4 and the anatomical body part 8. The wrap is preferably designed to include a compression feature, which in addition to other benefits can provide a tight interface between the wrap and the thermo-conductive layer. Such wraps include, for example, an additional outer layer for providing a compression fluid (i.e. air) to compress the wrap around the body part (as shown in FIG. 2C).

The system may further include a temperature sensor 10 located on or near the anatomical body part 8. The sensor output can be read by a controller 12 to provide temperature feedback to the heating or cooling unit 2.

The thermo-conductive material 6 can be any suitable material. For example, the thermo-conductive material may be a gel applied directly to the anatomical body part. Such gels are known in the art, and are used, for example, in cold “gel packs.” Numerous formulations of such gels are disclosed in U.S. Pat. No. 4,925,603, herein incorporated by reference. Applying the gel directly to the anatomical body part has many advantages. Specifically, the gel can be applied in such a way as to insure that any air gaps around the body part are adequately filled in, before the cooling wrap is placed on the body part.

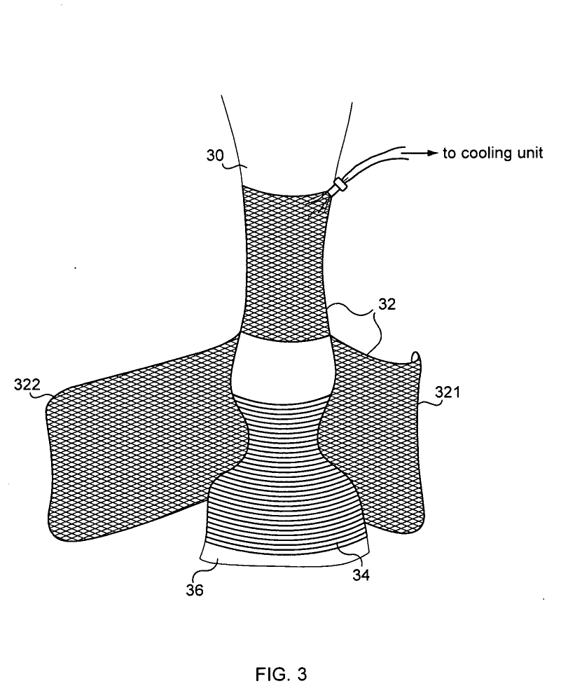

In another embodiment, the thermo-conductive material, such as a gel, may be encapsulated in a separate package, and placed around the anatomical body part. Additionally, the thermo-conductive material can be formed as an integral layer to the cooling wrap, as shown in FIGS. 2A, 2B and 2C. Specifically, in FIG. 2A, the cooling wrap may comprise cooling layer, and a thermo-conductive layer. As shown in FIG. 2B, the cooling wrap may comprise at least two thermo-conductive layers. As noted above and shown in FIG. 3B, the cooling wrap may also comprise an additional layer for providing compression to the wrap (i.e. air pressure in the layer).

For certain applications, the thermo-conductive material is preferably a “dry” material to eliminate clean-up issues associated with gels. The thermo-conductive material could also be fluids, such as water, saline solution, etc. Other examples include conformable releasable adhesives, special use plastics or rubber. These materials may or many not be encased in other materials as desired for particular applications.

In an alternate embodiment, additional thermo-conductive layers can be added to the system. For example, a cooling system could have two thermo-conductive layers as shown in FIG. 2B, where each thermo-conductive layer preferably has a different thermal conductivity and/or abilities to conform (pliability). The different layers may have different freezing points as well. This allows for further improved heat transfer from the cooling wrap to the anatomical body part. In addition, these additional layers can be the same form as the primary layer, or could be in a different form. In other words, in one particular application, a cooling wrap can be configured to have two integral thermo-conductive layers. In another application, a thermo-conductive gel may be applied directly to the body part, and a cooling wrap can be secured over the gel, wherein the cooling wrap has a single thermo-conductive layer integral to the wrap.

The present cooling system can be used to provide improved cold therapy to human and animal body parts. A thermo-conductive material can be applied to a body part, and then a cooling wrap can be placed over the thermo-conductive material. Of course, as noted above, the thermo-conductive material may be formed as an integral layer of the cooling wrap, as well. Cold fluid is then circulated through the cooling wrap from a cooling unit. The thermo-conductive material provides for heat conduction between the body part and the cooling wrap. This improves the ability of the cooling wrap to cool the body part, and maintain a more even and consistent temperature, thus resulting in improved cold therapy.

One specific application of the present invention is the treatment of equine laminitis. Laminitis is the most serious disease of the equine foot, and causes pathological changes in the anatomy that can lead to crippling changes in the foot. Currently, laminitis is the second leading cause of death in horses, and to date there is no cure. Recently, it has been shown that providing “cryotherapy” to the distal limbs of a horse during the early stages of laminitis, can reduce the severity of, or prevent laminitis. [See van Eps, A. W. and Pollitt, C. C., Equine laminitis. Cryotherapy Reduces the Severity of the Acute Lesion, Equine Vet. Journal, Vol. 36, pgs. 255-260 (2004)].

However, in order to provide cryotherapy to the equine foot, the temperature in the circulating fluid generally should be below 5° C., and preferably in the range of 5° and −5° C. In some recent studies, the temperature selected has been 1° C. and other equine therapeutic applications have used −4° C. For some applications, it may be desirable to have a temperature in the circulating fluid as low as −5° C. or even colder, in order to provide the desired temperature to the equine foot. Given the differences in size and shapes from horse to horse, it is extremely difficult to maintain a constant desired temperature at the hoof/foot of a horse.

Utilizing the present invention, a thermo-conductive gel can be applied directly to a horse's hoof and lower foot. A cooling wrap 32 can then be secured around the hoof and lower leg, as shown in FIG. 3. Specifically, a thermo-conductive gel 34 can be applied directly to the hoof/foot 36 of a horse. A cooling wrap can then be attached over the gel 34, and around the horse's lower leg 30. The flaps 321, 322 can be wrapped securely around the hoof and lower leg and secured with Velcro, or by similar means. Hoses from a cooling unit (not shown) are attached to the cooling wrap 32 to provide chilled fluid circulation through the cooling wrap 32.

The heat exchange fluid is chilled down to the desired temperature range by the cooling unit (such as 1°-5° C.). The thermo-conductive gel provides a very efficient heat conduction mechanism between the ultra-chilled fluid circulating in the wrap and the hoof and foot. Use of a cooling wrap alone does not provide satisfactory results, since much of the heat transfer is lost due to air gaps and imperfections in the fit of the wrap. The present invention thus provides a convenient and effective method to provide cryotherapy to treat laminitis in an equine foot.

As noted above, the gel can be applied directly to the horse's hoof, or it may be provided in a separate package, or integrated into the cooling wrap. Similarly, two or more layers of thermo-conductive material can be used, where each layer has a different thermal conductivity in order to improve the heat transfer. It is preferable that the thermo-conductive material is compliant at the desired cooling temperature i.e. does not freeze solid at the intended operating temperature.

Those skilled in the art will appreciate that various adaptations and modifications of the just-described preferred embodiments can be configured without departing from the scope and spirit of the invention. Therefore, it is to be understood that, within the scope of the appended claims, the invention may be practiced other than as specifically described herein.

What is claimed is:

1. A therapeutic cooling or heating system for cooling or heating an anatomical body part, the system comprising:
a wrap configured to conform to an anatomical body part, the wrap having one or more internal channels for fluid circulation;
a cooling or heating unit to provide cooled or warmed fluid to the wrap and to circulate the fluid through the one or more internal channels; and
at least one layer of a thermo-conductive material positioned between the wrap and the anatomical body part.

2. The therapeutic cooling or heating system of claim 1, wherein the thermo-conductive material is a gel applied directly to the anatomical body part.

3. The therapeutic cooling or heating system of claim 1, wherein the thermo-conductive material is contained in a protective covering separate from the cooling wrap.

4. The therapeutic cooling or heating system of claim 1, wherein the thermo-conductive material is integrally formed as a layer of the cooling wrap.

5. The therapeutic cooling or heating system of claim 1, wherein the at least one layer of thermo-conductive material comprises at least two layers of material, where each layer has a different thermal conductivity and/or pliability.

6. The therapeutic cooling or heating system of claim 1, wherein the cooling unit provides fluid at a temperature of between 5° and −5° C.

7. The therapeutic cooling or heating system of claim 6, wherein the thermo-conductive material is compliant at temperatures between 5° and −5° C.

8. The therapeutic cooling or heating system of claim 1, wherein the thermo-conductive material has a freezing point at least as low as −5° C.

9. The therapeutic cooling or heating system of claim 1, wherein the cooling or heating system unit provides chilled (or warmed) water to the wrap, and the thermo-conductive material provides a conductive heat exchange between the chilled (or warmed) water circulating through the wrap and the anatomical body part.

10. The therapeutic cooling or heating system of claim 1, wherein the thermo-conductive material provides conductive heat exchange between the wrap and the anatomical body part.

11. The therapeutic cooling or heating system of claim 1, wherein the wrap further comprises a compression layer to receive a compression fluid to compress the wrap against the anatomical body part.

12. The therapeutic cooling or heating system of claim 1, further comprising a temperature sensor located at or near the anatomical body part and a controller connected to receive an output from the temperature sensor, wherein the controller adjusts the temperature of the fluid based on the output received from the temperature sensor.

13. A method of providing cold or heat therapy to an anatomical body part, the method comprising:
placing a first thermo-conductive material on an anatomical body part;
placing a wrap around the thermo-conductive material; and
cooling or heating the wrap to efficiently add or remove thermal energy to the anatomical body part.

14. The method of claim 13, wherein the first thermo-conductive material is a gel placed directly on the anatomical body part.

15. The method of claim 13, wherein the first thermo-conductive material is contained in a protective covering separate from the wrap.

16. The method of claim 13, wherein the first thermo-conductive material is integrally formed as a layer of the wrap, and is placed next to the anatomical body part when placing the wrap.

17. The method of claim 13, further comprising placing a second thermo-conductive material between the anatomical body part and the wrap, wherein the first and second thermo-conductive materials have different thermal conductivities.

18. The method of claim 13, wherein the cooling fluid is at a temperature of between 5° and −5° C.

19. The method of claim 13, wherein the cooling fluid is chilled water.

20. The method of claim 13, further comprising providing a compression fluid to the wrap to compress the wrap around the anatomical body part.

21. A method of treating laminitis on a horse, the method comprising:
placing a thermo-conductive material on a horse's hoof;
placing a cooling wrap around the thermo-conductive material and a lower leg of the horse; and
providing a cooling fluid to the cooling wrap, wherein the cooling fluid has a temperature below 5° C.

22. The method of claim 21, wherein the thermo-conductive material is a gel placed directly on the horse's hoof and surrounding area.

23. The method of claim 21, wherein the cooling fluid has a temperature of approximately −4° C.

24. The method of claim 21, further comprising providing a compression fluid to the wrap to compress the wrap around the lower leg and hoof.

25. A therapeutic cooling or heating system for cooling or heating an anatomical body part, the system comprising:
a wrap configured to conform to an anatomical body part,
a cooling or heating unit to cool or heat the wrap; and
at least one layer of a thermo-conductive material positioned between the wrap and the anatomical body part.

26. The therapeutic cooling or heating system of claim 25, further comprising a temperature sensor located at or near the anatomical body part and a controller connected to receive an output from the temperature sensor, wherein the controller adjusts the temperature of the wrap based on the output received from the temperature sensor.
